(19) United States
(12) Patent Application Publication
(10) Pub. No.: US 2022/0163522 A1
(43) Pub. Date: May 26, 2022

(54) COMPOSITIONS AND METHODS FOR RAPID COVID-19 DETECTION

(71) Applicant: ARIZONA BOARD OF REGENTS ON BEHALF OF ARIZONA STATE UNIVERSITY, Scottsdale, AZ (US)

(72) Inventors: Chao WANG, Chandler, AZ (US); MD Ashif IKBAL, Tempe, AZ (US)

(73) Assignee: ARIZONA BOARD OF REGENTS ON BEHALF OF ARIZONA STATE UNIVERSITY, Scottsdale, AZ (US)

(21) Appl. No.: 17/532,969

(22) Filed: Nov. 22, 2021

Related U.S. Application Data
(60) Provisional application No. 63/116,953, filed on Nov. 23, 2020.

(51) Int. Cl. G01N 33/553; G01N 33/52; G01N 33/543

(52) U.S. Cl. CPC G01N 33/553; G01N 2333/165; G01N 33/54346; G01N 33/523

(57) ABSTRACT

The present disclosure provides compositions and methods related to COVID-19 detection. In particular, the present disclosure provides plasmonic metal nanoparticles (MNPs) for use in assays to detect and/or quantify neutralizing antibodies to SARS-CoV-2 in a sample. The compositions and methods of the present disclosure provide a portable, inexpensive, rapid, and accurate antibody assay platform that can be used to evaluate protective immune responses in individuals who have recovered from COVID-19 infection, as well as the efficacy, strength, and duration of vaccines that are under development or in clinical trials.

### CROSS-REFERENCE TO RELATED APPLICATIONS

This application claims priority to U.S. Provisional Patent Application Ser. No. 63/116,953, filed Nov. 23, 2020, the disclosure of which is incorporated herein by reference.

### FIELD

The present disclosure provides compositions and methods related to COVID-19 detection. In particular, the present disclosure provides plasmonic metal nanoparticles (MNPs) for use in assays to detect and/or quantify neutralizing antibodies to SARS-CoV-2 in a sample. The compositions and methods of the present disclosure provide a portable, inexpensive, rapid, and accurate antibody assay platform that can be used to evaluate protective immune responses in individuals who have recovered from COVID-19 infection, as well as the efficacy, strength, and duration of vaccines that are under development or in clinical trials.

### BACKGROUND

The new coronavirus disease (COVID-19), caused by the RNA virus severe acute respiratory syndrome coronavirus 2 (SARS-CoV-2), has infected >22 million people and caused >780,000 deaths in >210 countries, states, or territories, with >250,000 new cases daily. There are already >5.6 million cases and >175,000 deaths in the U.S., and these statistics continue to worsen. Given the high mortality rate (1-3% compared to 0.1% for influenza), fast transmission (reproductive number R0 is estimated 3-4 but significantly higher in densely populated areas), asymptotic infection in some individuals, relatively long hospitalization (˜2 weeks in average), and unavailable effective drugs or vaccines, COVID-19 has posed an unprecedented threat to human health and economics in the U.S. and the world.

Currently, clinical diagnosis of COVID-19 is mainly based on epidemiological history, clinical manifestations and biomolecular marker detection (e.g., real-time quantitative polymerase chain reaction). However, molecular diagnostic methods are not ideal for determining the transmission patterns and to calculate the burden of disease, or estimating the efficacy of donated convalescent plasma in treatment, or studying the duration and strength of immune response post-infection. Currently, most available serological tests that detect SARS-CoV-2 antibodies are lateral flow assays (LFA) that are based on simple positive or negative detection of antibodies, which is feasible for inexpensive and point-of-care (POC) use and large-scale surveillance but not informative regarding the amount, type, or function of the antibodies. An alternative for accurately detecting antibodies against SARS-CoV-2 is the enzyme-linked immunosorbent assay (ELISA), which can measure not only the presence but also the titer (amount) and type (IgG, IgM, IgA) of antibody. ELISA assays allow for a better measure of the strength of the humoral response, but are complex and can only be performed in a laboratory setting. Additionally, ELISA is not ideal for virus neutralization/blocking tests, which is crucially important in studying the humoral response during vaccine development and vaccination but not widely available. Current neutralization assays usually involve propagation of viruses and require such assays to be conducted in a biosafety level 3 (BSL3) lab settings, which unfortunately is unavailable to many researchers or the public.

### SUMMARY

Embodiments of the present disclosure include a composition comprising a first plurality of plasmonic metal nanoparticles (MNPs) having a SARS-CoV-2 antigen bound to its surface, and a second plurality of MNPs having an anti-IgG and/or an anti-IgM binding moiety bound to its surface. In accordance with these embodiments, the first and second pluralities of MNPs form a complex in the presence of a sample comprising a target antibody that recognizes the SARS-CoV-2 antigen.

In some embodiments, the first plurality of MNPs comprise gold, silver, copper, aluminum, platinum, and palladium, or any combinations thereof. In some embodiments, the first plurality of MNPs comprise a size and shape suitable for colorimetric, spectrometric, or electronic detection.

In some embodiments, the second plurality of MNPs comprise gold, silver, copper, aluminum, platinum, and palladium, or any combinations thereof. In some embodiments, the second plurality of MNPs comprise a size and shape suitable for colorimetric, spectrometric, or electronic detection.

In some embodiments, the SARS-CoV-2 antigen is bound to the first plurality of MNPs via a linker. In some embodiments, the anti-IgG and/or an anti-IgM binding moiety is bound to the second plurality of MNPs via a linker.

In some embodiments, the SARS-CoV-2 antigen comprises an S1 unit or receptor binding domain (RBD) of the spike (S) protein, or a fragment thereof.

In some embodiments, the composition is a liquid-phase composition.

In some embodiments, the composition further comprises a sample obtained from a subject's bodily fluid.

Embodiments of the present disclosure also include a method of performing a colorimetric, spectrometric, or electronic assay using any of the compositions described herein. In accordance with these embodiments, the method comprises combining the first and second pluralities of MNPs with the sample from a subject, and detecting an altered MNP extinction wavelength corresponding to the first and/or second pluralities of MNPs based on the presence or absence of the target antibody.

Embodiments of the present disclosure also include a system for performing any of the colorimetric, spectrometric, or electronic assays described herein. In accordance with these embodiments, the system comprises a receptacle for combining the first and second pluralities of MNPs with the sample from a subject, a light source capable of emitting an MNP extinction wavelength corresponding to the first and/or second pluralities of MNPs, and a photodetector capable of detecting transmitted light from the first and/or second pluralities of MNPs.

In some embodiments, the system further comprises a means for determining a voltage and/or current readout corresponding to the transmitted light detected by the photodetector.

Embodiments of the present disclosure also include a composition comprising a first plurality of plasmonic metal nanoparticles (MNPs) having a SARS-CoV-2 antigen bound to its surface, and a second plurality of MNPs having a SARS-CoV-2 antigen binding moiety bound to its surface. In accordance with these embodiments, the first and second pluralities of MNPs form a complex in the absence of a sample comprising a target antibody that recognizes the SARS-CoV-2 antigen.

In some embodiments, the first plurality of MNPs comprise gold, silver, copper, aluminum, platinum, and palladium, or any combinations thereof. In some embodiments, the first plurality of MNPs comprise a size and shape suitable for colorimetric, spectrometric, or electronic detection.

In some embodiments, the second plurality of MNPs comprise gold, silver, copper, aluminum, platinum, and palladium, or any combinations thereof. In some embodiments, the second plurality of MNPs comprise a size and shape suitable for colorimetric, spectrometric, or electronic detection.

In some embodiments, the SARS-CoV-2 antigen is bound to the first plurality of MNPs via a linker. In some embodiments, the SARS-CoV-2 antigen binding moiety is bound to the second plurality of MNPs via a linker.

In some embodiments, the SARS-CoV-2 antigen comprises an S1 unit or receptor binding domain (RBD) of the spike (S) protein, or the S protein, or a fragment thereof. In some embodiments, the SARS-CoV-2 antigen binding moiety comprises the angiotensin-converting enzyme 2 (ACE2), or a fragment thereof.

In some embodiments, the composition is a liquid-phase composition.

In some embodiments, the composition further comprises a sample obtained from a subject's bodily fluid.

Embodiments of the present disclosure also include a method of performing a colorimetric, spectrometric, or electronic assay using any of the compositions described herein. In accordance with these embodiments, the method comprises combining the first and second pluralities of MNPs with the sample from a subject, and detecting an altered MNP extinction wavelength corresponding to the first and/or second pluralities of MNPs based on the presence or absence of the target antibody.

Embodiments of the present disclosure also include a system for performing the any of the colorimetric, spectrometric, or electronic assays described herein. In accordance with these embodiments, the system comprises a receptacle for combining the first and second pluralities of MNPs with the sample from a subject, a light source capable of emitting an MNP extinction wavelength corresponding to the first and/or second pluralities of MNPs, and a photodetector capable of detecting transmitted light from the first and/or second pluralities of MNPs.

In some embodiments, the system further comprises a means for determining a voltage and/or current readout corresponding to the transmitted light detected by the photodetector.

Embodiments of the present disclosure include a composition comprising a plurality of plasmonic metal nanoparticles (MNPs) having a SARS-CoV-2 antigen bound to its surface, wherein the plurality of MNPs form a complex in the presence of a sample comprising a target antibody that recognizes the SARS-CoV-2 antigen.

In some embodiments, the plurality of MNPs comprise gold, silver, copper, aluminum, platinum, and palladium, or any combinations thereof. In some embodiments, the plurality of MNPs comprise a size and shape suitable for colorimetric, spectrometric, or electronic detection.

In some embodiments, the SARS-CoV-2 antigen is bound to the plurality of MNPs via a linker.

In some embodiments, the SARS-CoV-2 antigen comprises an S1 unit or the receptor binding domain (RBD) of the spike (S) protein, or a fragment thereof.

In some embodiments, the composition is a liquid-phase composition.

In some embodiments, the composition further comprises a sample obtained from a subject's bodily fluid.

Embodiments of the present disclosure also include a method of performing a colorimetric, spectrometric, or electronic assay using any of the compositions described herein. In accordance with these embodiments, the method comprises combining the plurality of MNPs with the sample from a subject, and detecting an altered MNP extinction wavelength corresponding to the plurality of MNPs based on the presence or absence of the target antibody.

Embodiments of the present disclosure also include a system for performing any of the colorimetric, spectrometric, or electronic assays described herein. In accordance with these embodiments, the system comprises a receptacle for combining the plurality of MNPs with the sample from a subject, a light source capable of emitting an MNP extinction wavelength corresponding to the plurality of MNPs, and a photodetector capable of detecting transmitted light from the plurality of MNPs.

In some embodiments, the system further comprises a means for determining a voltage and/or current readout corresponding to the transmitted light detected by the photodetector.

### BRIEF DESCRIPTION OF THE DRAWINGS

FIGS. 1A-1F: Colorimetric, spectrometric, or electronic sensing mechanism using protein- and antibody-coated metal nanoparticles and two different assay formats (sandwich and competitive) for COVID-19 antibody sensing (A-C). Portable readout system, including bare-eye reading, spectroscopic analysis using a polydimethylsiloxane (PDMS) well plate, and electronic readout with light-emitting diodes (LEDs) and photodetectors (D-F). (Data shown in D and E are from preliminary work using Ebola virus protein sensing.)

FIG. 2: Schematic diagram of SARS-CoV-2 virion particle and RBD-ACE2 binding.

FIGS. 3A-3C: Data of colorimetric sensing of IgG. Schematics showing the sensing mechanism using AuNPs surface-conjugated with spike-RBD (A-B). Optical image showing the feasibility of detecting IgG with bare-eye after incubation for ˜3 hours (C).

FIGS. 4A-4D: Preliminary data of spectrometric IgG sensing in PDMS well plate. Schematics of measurement setup (A). Optical image of AuNP assay in PDMS well plate bonded to glass (B). Measured extinction spectra of PDMS well plate (C). The extracted extinction signal intensity (black) at ˜560 nm compared to ELISA (grey) (D).

FIGS. 5A-5I: Exemplary results to structurally and optically characterize the AuNPs on a surface in Ebola protein sensing. Cryo-TEM image of: (A) 80 nm antibody-coated AuNP suspension without target Ebola sGP proteins, and (B) AuNP aggregate with 1 nM Ebola sGP proteins. Optical image of suspension supernatant dried on glass slide (C). Optical image of suspension supernatant on gold in wet state (the dried sample has low contrast to distinguish the different spots due to high reflectivity from gold) (D). Extinction spectra of dried sample on glass at different IgG concentrations (E). SEM images showing the change of AuNPs with IgG concentration (F). The extracted extinction signal from FIG. 5E (G). AuNP density on gold substrate from SEM images in FIG. 5F (H). Dark-field scatter spot counting on gold substrate (I).

FIGS. 6A-6L: Exemplary results of studying the effect of MNP size on spectrometric sensing Ebola sGP proteins in PDMS well plate. Sensing using 40 nm diameter AuNPs: Optical image of PDMS plate (A), Measured spectra (B), Standard sensing curve of extinction signals versus sGP concentration (C), and the extinction signals versus incubation time (D). Sensing using 80 nm diameter AuNPs (E-H). Sensing using 100 nm diameter AuNPs (I-L). The dynamic plots are for detecting 10 nM sGP in 1×PBS.

FIGS. 7A-7C: Model in understanding MNP colorimetric sensing mechanism. Schematic of ligand-coated MNPs contributing to extinction (A). Schematic of clustered MNPs sediment that decreases the active monomer concentration and the extinction (B). A mathematically calculated sensing standard curve using 80 nm AuNPs (C).

FIGS. 8A-8F: Anisotropic MNPs for colorimetric sensing. Calculated transmission spectra of gold and silver NRs with different aspect ratios (AR from 1.4 to 3.5) (A-B). The expected suspension color for the different Ars (C-D). A CMYK color mixing model with arrows indicating the colors from the simulations (E). An exemplary color mixing scheme assuming magenta, cyan and yellow (shown in greyscale) as the primary suspension colors (F).

FIGS. 9A-9D: Schematic diagram of sandwich-type assay for immunoglobulin sensing. Schematic of RBD-conjugated MNPs to detect total immunoglobulins (A-B). Schematics showing RBD-conjugated and anti-IgG (or anti-IgM) coated heterogeneous MNPs to selectively detect IgG or IgM (C-D). The MNP clustering is expected to reduce the extinction signals from both the NPs and NRs and thus decrease the assay suspension color intensity.

FIGS. 10A-10C: Schematic diagram of competitive assay for nAb sensing. Schematic of RBD- and ACE2-conjugated MNPs to form clusters from the RBD-ACE2 binding without nAb (A). The MNP molar ratio can be adjusted to display initial color (e.g., light red with excess AuNPs). Schematics of adding nAbs to form RBD-nAbs complex and release ACE2-coated NRs to suspension (B-C). The color and intensity changes indicate the effectiveness of the nAbs.

FIGS. 11A-11G: Exemplary results of RBD-binding antibody (Ab) testing with spectrometric readout.

FIGS. 12A-12H: Exemplary data from neutralization testing with spectrometric readout.

FIGS. 13A-13F: Exemplary results of rapid sensing of Ebola sGP proteins. Schematic showing fast AuNP crosslinking mediated by centrifugation (A). Optical image of PDMS well plate in detecting sGP with concentrations from 1 pM to 1 μM in 1×PBS (B). This was after centrifugation, 20-min incubation and vortex mixing. Black paper was attached to the plate to minimize background optical noise. Extinction spectra of AuNP assay (C). Extinction maximum plotted as standard sensing curve (D). Optical images and extinction signals to verify the influence of incubation time on extinction signals. (E-F). Inset shows sensing data displayed in a narrowed signal range.

FIGS. 14A-14D: Portable electronic readout system. Schematic showing the key components in electronic sensing (A). Optical images showing welded LED and photodetectors and a schematic of a 3D-printed microcentrifuge tube holder (B). Optical image showing the voltage readout on a multi-meter from a resistor in series with the photodetector (C). Measured photocurrent versus reverse bias voltage in detection of AuNPs of different concentrations as well as PBS buffer as the background (D).

### DETAILED DESCRIPTION

The new coronavirus disease (COVID-19), caused by the RNA virus severe acute respiratory syndrome coronavirus 2 (SARS-CoV-2), in the U.S. alone has infected more than 5.6 million people, caused ˜175,000 deaths, and resulted in loss of more than 10 million jobs and trillions of dollars. Serology tests that detect antibodies responsive to the infection have emerged as a valuable tool to assist virus surveillance, assess the risks of infection, evaluate the quality of convalescent plasma donation, and study the duration and magnitude of immune response post-infection. Many of the available tests are lateral flow assays (LFA) or enzyme-linked immunosorbent assays (ELISA). LFA provides simple positive or negative results, and are feasible for point-of-care (POC) use but less useful to identify the amount, type, or function of the antibodies. ELISA provides a better quantification, but it is not suitable for rapid testing due to its complexity in operation and requirement of laboratory instruments.

Embodiments of the present disclosure provide plasmonic metal nanoparticle (MNP) based colorimetric, spectrometric, or electronic assays to identify and quantify COVID19-related antibodies using optical and electronic readouts. Analytes (e.g., immunoglobulins including neutralizing antibodies) modulate the extent of MNP clustering and precipitation, and accordingly, changes the suspension color and intensity, which can be quantified to determine the concentration, binding affinity, and even binding epitope of the analyte. The present disclosure has the capability to substantially promote the availability of serology tests and assist the diagnosis, vaccination, and treatment of COVID-19 disease.

In accordance with this, embodiments of the present disclosure provide a portable colorimetric, spectrometric, or electronic sensor design for rapid detection of COVID-19 antibodies, including different types of immunoglobulins (IgG, IgM, IgA, etc.) and virus-specific neutralizing antibodies (nAbs). In some embodiments, different assay variants can be used, including MNP in suspension and dried states (bare-eye readout), spectroscopic quantification, and optical and structural analysis. In some embodiments, the MNP shape and size, analyte and MNP concentration, and binding affinity affect the limit of detection, dynamic range, and assay time will be incorporated into the assays of the present disclosure. Additionally, immunoglobulin and virus-specific ligands can be conjugated, such as anti-IgG (or anti-IgM), the receptor-binding domain (RBD) from SARS-CoV-2 spike protein, and peptide ligands derived from nAb epitope characterization studies, on MNPs of different geometries and materials that display distinct colors. Such heterogeneous MNPs can be used to establish a sandwich-type assay capable of detecting multiple types of antibodies by bare eyes. Additionally, a competitive assay can be developed that includes heterogeneous MNPs surface-conjugated with RBD and human angiotensin-converting enzyme 2 (ACE2), a cell receptor responsible for SARS-CoV-2 infection. Effective nAbs compete with ACE2-bound MNPs in RBD binding to prevent the clustering of such MNPs, while ineffective nAbs cause MNP precipitation and change in the assay color. As would be recognized by one of ordinary skill in the art based on the present disclosure, the compositions, assays, and systems described herein can be used with any SARS-CoV-2 antigen recognized by antibodies in a sample. In some aspects, the present disclosure includes references to the analysis and detection of Ebola virus proteins (e.g., sGP). These aspects are included, for example, to further illustrate certain general principles of the assay formats disclosed herein that are optionally adapted for use in the analysis and detection of COVID-19 antibodies.

Section headings as used in this section and the entire disclosure herein are merely for organizational purposes and are not intended to be limiting.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain (s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Correlated to” as used herein refers to compared to.

The term “derived from” as used herein refers to cells or a biological sample (e.g., blood, tissue, bodily fluids, etc.) and indicates that the cells or the biological sample were obtained from the stated source at some point in time. For example, a cell derived from an individual can represent a primary cell obtained directly from the individual (e.g., unmodified). In some instances, a cell derived from a given source undergoes one or more rounds of cell division and/or cell differentiation such that the original cell no longer exists, but the continuing cell (e.g., daughter cells from all generations) will be understood to be derived from the same source. The term includes directly obtained from, isolated and cultured, or obtained, frozen, and thawed. The term “derived from” may also refer to a component or fragment of a cell obtained from a tissue or cell, including, but not limited to, a protein, a nucleic acid, a membrane or fragment of a membrane, and the like.

The term “isolating” or “isolated” when referring to a cell or a molecule (e.g., nucleic acids or protein) indicates that the cell or molecule is or has been separated from its natural, original or previous environment. For example, an isolated cell can be removed from a tissue derived from its host individual, but can exist in the presence of other cells (e.g., in culture), or be reintroduced into its host individual.

As used herein, the term “severe acute respiratory syndrome coronavirus-2” or “SARS-CoV-2” refers to the coronavirus that emerged in 2019 to cause a human pandemic of an acute respiratory disease, now known as coronavirus disease 2019 (COVID-19).

As used herein, the term “subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (e.g., a monkey, such as a cynomolgus or rhesus monkey, chimpanzee, etc.) and a human). In some embodiments, the subject may be a human or a non-human. In one embodiment, the subject is a human. The subject or patient may be undergoing various forms of treatment.

As used herein, the term “treat,” “treating” or “treatment” are each used interchangeably herein to describe reversing, alleviating, or inhibiting the progress of a disease and/or injury, or one or more symptoms of such disease, to which such term applies. Depending on the condition of the subject, the term also refers to preventing a disease, and includes preventing the onset of a disease, or preventing the symptoms associated with a disease (e.g., viral infection). A treatment may be either performed in an acute or chronic way. The term also refers to reducing the severity of a disease or symptoms associated with such disease prior to affliction with the disease. Such prevention or reduction of the severity of a disease prior to affliction refers to administration of a treatment to a subject that is not at the time of administration afflicted with the disease. “Preventing” also refers to preventing the recurrence of a disease or of one or more symptoms associated with such disease.

### 2. Detection Assays Using Metal Nanoparticles

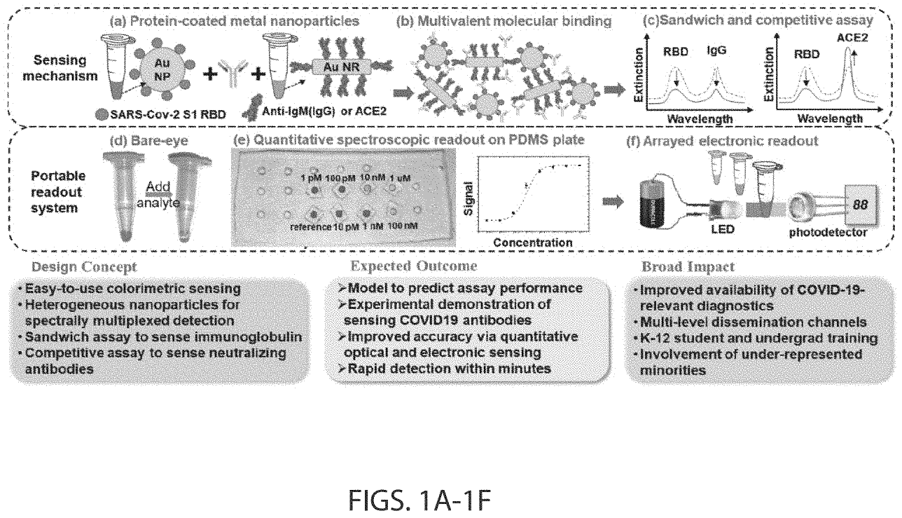

Embodiments of the present disclosure also include a new plasmonic metal nanoparticle (MNP) based colorimetric, spectrometric, or electronic assay platform that will support a variety of sensing schemes, including multiplexed detection of SARS-CoV-2 immunoglobulins and validating the efficacy of potent nAbs (FIG. 1). Using different assay variants, such as MNP in suspension (e.g., in microcentrifuge tubes or customized PDMS well plate) and dried states (e.g., on glass or gold surface), structural analysis and optical detection are combined with intuitive physical pictures and a theoretical mathematical model to comprehensively understand the mechanisms of MNP-based multivalent analyte-binding in antibody sensing. Such studies will build a foundation to further incorporate heterogeneous MNPs displaying distinct colors from blue to red to improve specificity, achieve multiplexed detection, and expand assay functionalities. In addition, a portable and inexpensive detecting instrument will be developed that provides more precise quantification than bare-eye readout, feasible for clinical settings and field deployment.

Embodiments of the present disclosure enable a comprehensive understanding of antibody-sensing mechanisms using MNP-based colorimetric, spectrometric, or electronic assays, experimentally determined assay performance, and a complete suite of antibody sensing solution without requiring lab instruments or personnel training. The assay platform provided herein will facilitate inexpensive, fast, and accurate antibody detection/quantification that can evaluate protective immune responses in individuals who have recovered from COVID-19 infection or who are at high risk of new infection. These assays can be used to evaluate the efficacy, strength, and duration of vaccines that are under development or in clinical trials. Additionally, understanding MNP-based sensing mechanisms will establish functional assay formats and demonstrated sensor performance, which will serve to accelerate the design of other POC tests for diagnosis and treatment of COVID-19 disease.

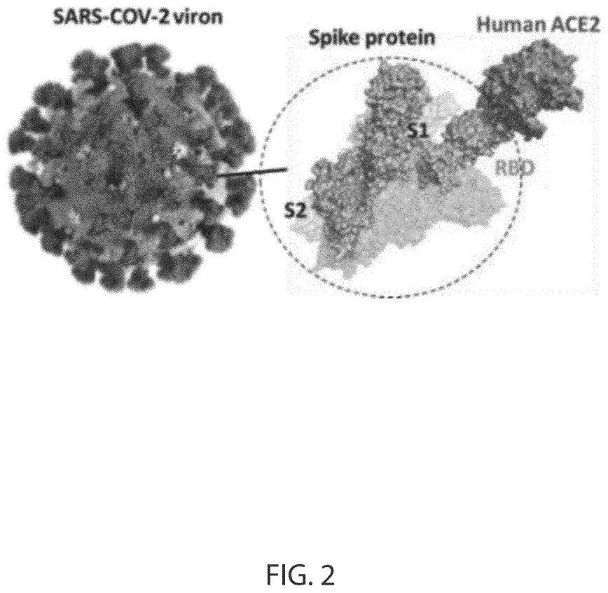

SARS-CoV-2 virions are spherical nanoparticles of about 100 nm with a membrane envelope that is studded with homotrimers of the spike (S) glycoprotein (FIG. 2). S proteins are post-translationally cleaved in the secretory pathway to yield N- and C-terminal S1 and S2 subunits, respectively. S1 is organized into an N-terminal domain (NTD), a central receptor-binding domain (RBD), and a C-terminal domain (CTD). The S1 RBD engages the viral receptor, human angiotensin-converting enzyme 2 (ACE2), at the host cell surface, followed by S protein cleavage by the transmembrane protease serine protease-2 (TMPRSS2) at the cell surface, as well as in endosomes. This cleavage activates S2 conformational rearrangements that catalyze the fusion of viral and cellular membranes and escape of the viral genome into the cytoplasm, which initiates disease-causing cycles of viral replication. Following infection, most individuals will develop an immune response to the virus, including the production of neutralizing antibodies that can prevent future infection by blocking the binding activity of the S glycoprotein. Therefore, the S glycoprotein is the major antigenic target on the virus for protective antibodies, and is thus of high significance for diagnostics as well as the development of vaccines and therapeutic antibodies. Current COVID-19 diagnosis is mainly based on epidemiological history, clinical manifestations and biomolecular marker detection. At present, real-time quantitative polymerase chain reaction (RT-qPCR) that identifies the viral RNA SARS-CoV-2 is most widely used. Yet, the PCR assay does not provide information regarding the immune response.

Antibody-based detection, such as by enzyme-linked immunosorbent assay (ELISA), has shown the feasibility of detecting IgM and IgG antibodies in serum, which indicate the short-term and long-term immune response to pathogens. Studies with SARS-CoV-2 and other human CoVs demonstrate a marked transition from seronegative to seropositive for both Ig and IgM occurs about 9 days after the onset of symptoms. These serological tests, although not ideal for early detection of viral infection, serve to identify recent and past infections and to conduct population-level surveillance, which is critical to understanding the transmission, pathogenesis, mortality rate, and epidemiology of SARS-CoV-2 viruses. Many of the commercially approved tests are lateral flow assays (LFA), which involves running the fluid containing antibodies (patient blood) over a solid substrate containing SARS-CoV-2 antigens. If the antibodies are present in the blood, they will bind the viral protein and cause a color change indicating a positive test. The LFA test, based on simple positive or negative detection of antibodies, is useful for large scale surveillance, but does not provide any information regarding the amount, type, or function of the antibodies. A better test for accurately detecting antibodies against SARS-CoV-2 is the enzyme-linked immunosorbent assay (ELISA), a common laboratory test that can measure not only the presence but also the titer (amount) and type (IgG, IgM, IgA) of antibody. This test allows for a better measure of the strength of the humoral response. In general, the higher the antibody titer the better the protection. However, the ELISA assays are more complex and can only be performed in a laboratory setting but not POC use.

Currently, a number of promising vaccines are under active development and in clinical trials. For most of them, the key is to train the immune system to generate neutralizing antibodies (nAbs) that recognize SARS-CoV-2's S protein and block its cellular entry via binding to the ACE2 cell receptor. Indeed, plasma derived from human convalescents and replete with nAbs has shown early promise as a COVID-19 treatment. The quality and quantity of the antibody response dictate functional outcomes. For example, in the case of SARS-CoV, viral docking on ACE2 on host cells is blocked when nAbs recognize the RBD domain or the heptad repeat 2 (HR2) domain on the S protein. In addition, nAbs can interact with other immune components, including phagocytes and natural killer cells, to assist pathogen clearance. However, sub-optimal pathogen-specific antibodies can promote pathology in some cases, resulting in a phenomenon known as antibody-dependent enhancement (ADE). Multiple factors determine whether an antibody neutralizes a virus or causes ADE and acute inflammation, including the specificity, concentration, affinity and isotype of the antibody. For example, in vitro data suggest that ADE occurs when antibody is present at a low concentration but dampens at the high-concentration range.

Although vaccines encoding SARS-CoV S protein and nucleocapsid (N) protein both provoke anti-S and anti-N IgG in immunized mice to a similar extent, N protein-immunized mice show significant upregulation of pro-inflammatory cytokine secretion and more severe lung pathology. Similarly, antibodies targeting different epitopes on S protein may vary in their potential to induce neutralization or ADE. For instance, antibodies reactive to the RBD domain or the HR2 domain of the S protein induce better protective antibody responses in non-human primates, whereas antibodies specific for other S protein epitopes can induce ADE. It is reported that the recombinant SARS-CoV-2 RBD antigen is highly sensitive and specific for detection of antibodies induced by SARS-CoVs. Further, a strong correlation was observed between the levels of RBD-binding antibodies and levels of SARS-CoV-2 neutralizing antibodies in patients. It was also found that only RBD-binding nAbs showed SARS-CoV-2 pseudovirus neutralization effects, and only nAbs bound to the RBD with a kD smaller or close to the dissociation constant of ACE2/RBD (15.9 nM) would have significant neutralization effects. These results support the use of RBD-based antibody assays for serology and as a correlate of neutralizing antibody levels in people who have recovered from infections or vaccinated. In addition to targeting the RBD of SARS-CoV-2, the compositions, assays, and systems described herein can be used with any SARS-CoV-2 antigen recognized by antibodies in a sample, as would be recognized by one of ordinary skill in the art based on the present disclosure.

Neutralizing assay is important toward evaluating the effectiveness of nAbs in blocking the viral infection. The gold standard is viral plaque reduction neutralization assay, where viruses replicate inside cells grown in cultures and are subsequently released when the cells are lysed or killed. This assay measures not only the titer of the antibody but also its ability to protect against viral infection. However, these assays are very labour intensive and must be performed in biosafety level 3 (BSL3) labs. Given limited access to BSL-3 facilities, researchers have turned to surrogate viral systems. These include retroviruses, lentiviruses, or replication-defective pseudoviruses with SARS-CoV-2 S protein and other molecular competent for a single round of viral entry and infection. However, these pseudotyped viruses are typically laborious to produce and challenging to scale up. Currently, there is still a lack of easy-to-use, inexpensive and accurate neutralization assays, which are important for drug discovery, vaccine development, and patient treatment with donated convalescent plasma.

Based on the above, embodiments of the present disclosure combine experimental analysis with simple physical interpretations and a theoretical model to comprehensively study the mechanisms of MNP-based multivalent analyte-binding in antibody sensing, and evaluate the assay performance in limit of detection, specificity, assay time, etc. At least two assays comprising heterogeneous MNPs are provided, including a sandwich assay for immunoglobulin sensing and a competitive assay for nAb sensing. Detection systems with portable electronic readout capability can be used with both assays, and these systems will incorporate different assay variants (e.g., MNP in liquid phase (in microcentrifuge tubes or customized polydimethylsiloxane (PDMS) well plate) and dried state (on glass or gold surface), for bare-eye readout, spectroscopic quantification, and optical and structural analysis).

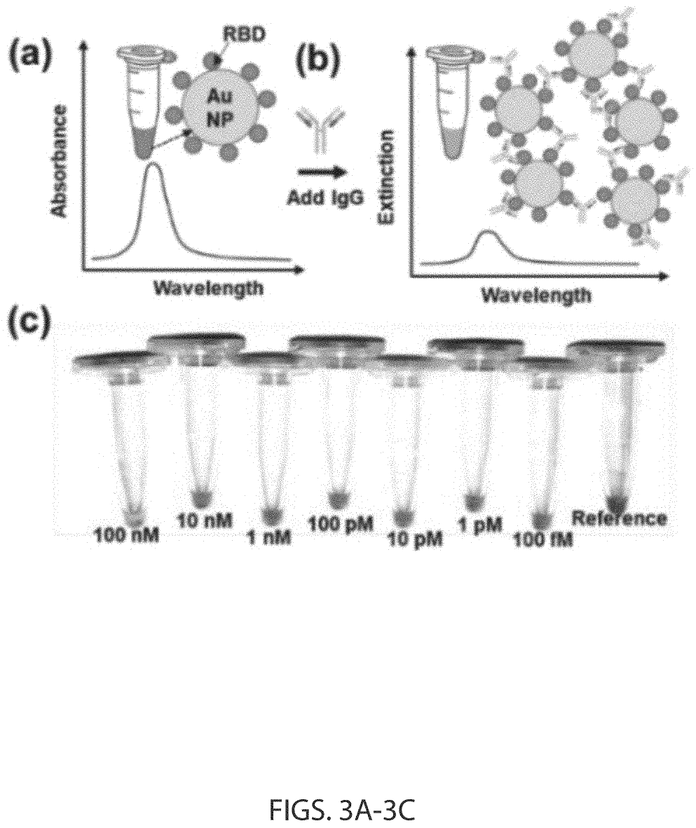

Embodiments of the present disclosure include a liquid-phase sensing system to detect antibody-induced MNP concentration changes. Using gold nanoparticles (AuNPs) as an example (FIGS. 3A-3B), the AuNP monomers are initially uniformly dispersed, presenting a reddish color of the suspension in a microcentrifuge tube due to extinction from LSPR resonance (e.g., at around 560 nm for 80 nm particles). In one embodiment, the AuNPs can be surface-coated with streptavidin by first self-assembly thiolated carboxyl poly(ethylene glycol) linker via thiol-sulfide reaction and then functionalization of streptavidin via amine-carboxyl coupling by N-Hydroxysuccinimide/1-Ethyl-3-(3-dimethylaminopropyl) carbodiimide (NHS/EDC) chemistry. Then, biotinylated RBD will be mixed with AuNPs, followed by filtration to remove excessive RBD, to form the RBD rendered AuNPs for detection of virus-binding antibodies. This suspension of AuNPs is ready to detect immunoglobulins such as IgG via IgG-RBD binding, resulting in subsequent bridging of AuNP monomers to larger aggregates. These AuNP aggregates eventually precipitate driven by gravity. As a result, higher IgG concentrations would result in smaller concentration of AuNP monomers in the suspension and significant decrease in color intensity (or saturation) (FIG. 3C). For example, the color contrast between 100 nM or 10 nM IgG samples and a reference (PBS buffer in lieu of IgG) can be easily recognized by naked eye or imaging (e.g., using a smartphone).

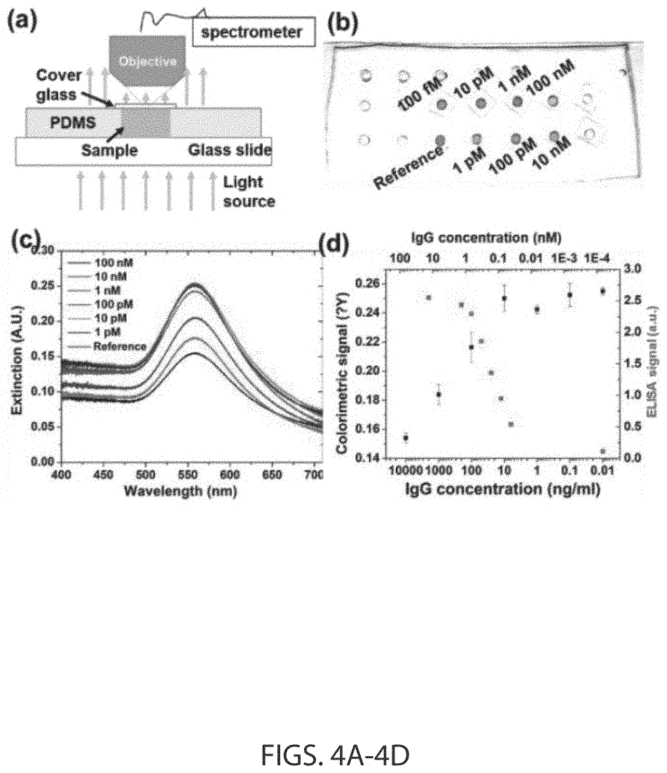

Naked eye readout is very useful for semi-quantitative diagnostics, but more accurate quantification requires more careful analysis of the optical spectra of the assay liquid. Embodiments of the present disclosure include the use of a customized PDMS well plate as a sample cuvette to obtain improved accuracy. The PDMS wells can be designed into different thicknesses (through curing in a petri-dish) and diameters (by punchers) and bonded to a glass slides after solvent cleaning and oxygen plasma treatment. This well plate can be sealed with a cover glass to avoid solution evaporation, and readily examined using a UV-visible spectrometer coupled to an upright microscope for spectral readout (FIG. 4A).

For example, a 2 mm-diameter and 3-mm thick PDMS well plate was pipette-loaded with ˜5 μL supernatant extracted from an assay suspension for IgG detection (FIG. 4B). Noticeably, the color contrast in the PDMS well plate was high enough in distinguishing ˜10 nM (˜10³ ng/ml) and higher IgG concentration from the reference sample. The PDMS well plate was examined under 50× objective for UV-visible spectra measurement (FIG. 4C), and the spectral intensity at the extinction peak position (˜559 nm for 80 nm AuNP assay) was plotted against the IgG concentration as a standard sensing curve (black dots in FIG. 4D). According to Beer-Lambert law A=εcl (A extinction signal, ε extinction coefficient, c concentration, l optical path), the decrease in extinction (FIGS. 4C-4D) corresponded to decrease in available 80 nm AuNP monomers in suspension at higher analyte concentration, consistent with the proposed sensing mechanism (FIGS. 3A-3B). This spectral analysis showed a dynamic range of at least 3 decades from >100 nM (˜10⁴ ng/ml) to ˜100 pM (˜10 ng/ml). In comparison, commercially available ELISA (grey dots in FIG. 4D) revealed a similar detection limit but a much more limited dynamic range of smaller than 2 decades from ˜200 ng/ml to <10 ng/ml. Noticing that the IgG concentration in serum of mild COVID-19 patients has been reported in the range of 10²-10^{4.5} ng/ml, these assays can outperform ELISA in COVID-19 antibody sensing with a much simpler assay format.

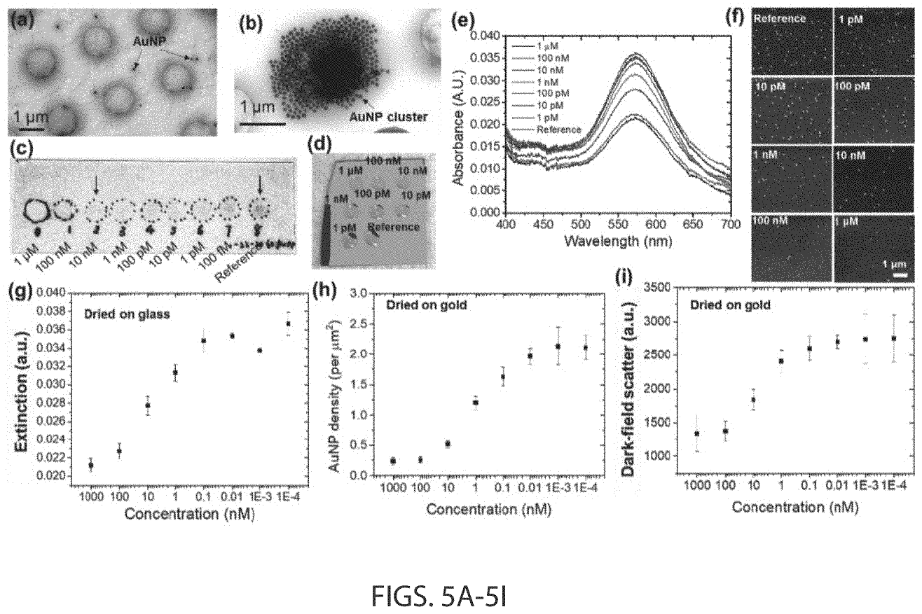

Experiments were also performed to test the optical and structural features of MNP assays in solid phase. To assist the microscopic-scale understanding of the analyte-ligand-MNP binding (FIGS. 3 and 4) and guide further optimization of the assay performance, a suite of solid-phase characterization methods that measure the MNP structures and quantities were developed (FIG. 5). MNP morphology of the precipitate will be analyzed from the microcentrifuge tubes containing the analyte (IgG and IgM) under cryogenic transmission electron microscope (Cryo-TEM, Titan Krios) (FIGS. 5A-5B). Initial data showed that only AuNP monomers were observed for the sample without sGP (FIG. 5A). In comparison, aggregation and clustering of 80 nm AuNP were observed at the presence of 10 nM sGP (about 2.5 by 1.9 μm in FIG. 5B).

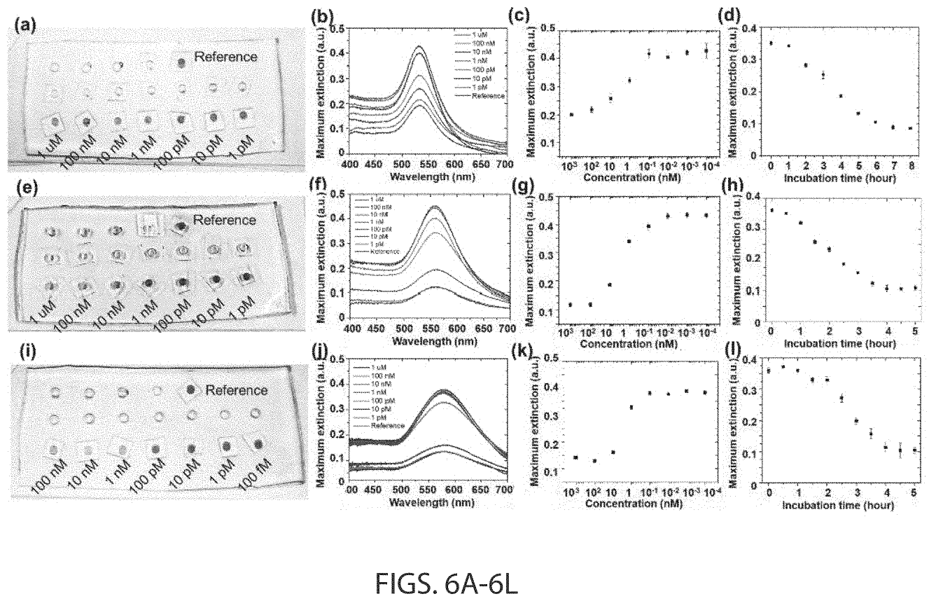

Additionally, MNPs will be analyzed by drop-casting a small volume (˜1 μL) of the AuNP assay supernatant and drying on a solid substrate. Previously, glass slides (FIG. 5C) and gold-coated silicon wafers (FIG. 5D) were used in Ebola protein sensing. The colors of dried sample spots on glass were visibly distinguishable, from transparent at high sGP concentration to red at low sGP concentration. This was consistent with extinction spectral measurement (FIG. 5E), which displayed sGP-dependent AuNP LSPR spectral modulation similar to solution measurement in PDMS well plate (FIG. 4C) but with about 10 times smaller signals. This signal decrease was attributed to significantly smaller amount of AuNPs in the optical path (estimated ˜300 μm thick, ˜1 μL) compared to the PDMS well plate (˜3 mm, ˜12 μL in total volume). In comparison, the samples dried on Au films (FIG. 5D) would not show such an optical contrast due to the high reflectivity of gold, but were instead perfect for SEM imaging (FIG. 5F) and dark-field optical imaging. The SEM images clearly showed decreasing AuNPs in the supernatant at high sGP concentrations, confirming AuNP precipitation during the 3-hour incubation period. Additionally, the standard sensing curve was plotted using the extracted extinction spectral peak intensity (FIG. 5G), the SEM-measured AuNP surface density (FIG. 5F) and count of dark-field scatters (bright spots in images) (FIG. 5I). The data showed that all three methods yielded consistent sensing results comparable to Ebola protein sensing in microcentrifuge tubes and PDMS well plates (FIG. 6), with a detection limit about 100 pM and a dynamic range of about 3-4 decades.

The size and shape of MNPs play important roles in colorimetric, spectrometric, or electronic sensing, because they not only determine the optical resonance and thus the observed color but also directly affect the sensitivity and assay time. Previously, the size effect in sensing of Ebola sGP proteins were studied (FIG. 6). Clearly, the color of the assay is redder for small particles but greener for larger particles (FIGS. 6A, 6E, and 6I (shown in greyscale)), attributed to redshift in LSPR resonance wavelengths at larger NP sizes (FIGS. 6B, 6F, and 6J). For bare-eye colorimetric imaging, one also needs to consider the spectral sensitivity of human eyes, which is centered around 555 nm in normal light settings. However, spectrum-based optical or electronic readout is not constrained by such a limitation.

Additionally, the effect of NP size on the assay sensitivity and sensing time was investigated (FIGS. 6C-6D, 6G-6H, 6K-6L). The starting AuNP suspension were kept at same optical density level at their peak resonance wavelength (533, 559 and 578 nm for 40, 80 and 100 nm diameter), at an AuNP concentration [MNP] of 0.275, 0.036 and 0.019 nM, respectively. Indeed, the extinction coefficient of MNPs is theoretically proportional to the total mass (or volume) of MNPs as σₑₓₜ∝[MNP]d³, therefore [MNP] drops with the particle diameter given a fixed total extinction. There are a few interesting observations. First, the detection limit was about 10-100 pM for all particle diameters, with the best about 10 pM for 80 nm NPs. Second, the dynamic range is size dependent, about 4 decades for 40 nm and 80 nm NPs but only ˜2 decades for 100 nm NPs. Further, the incubation time was much longer for small NPs (8 h for 40 nm) than larger particles (3 h for 80 and 100 nm AuNP assays) to achieve comparable color contrast (defined as A_{reference}/A_{10 nM}). These results helped to identify 80 nm AuNPs as the best candidate for a higher sensitivity, a broader dynamic range, and a shorter assay time.

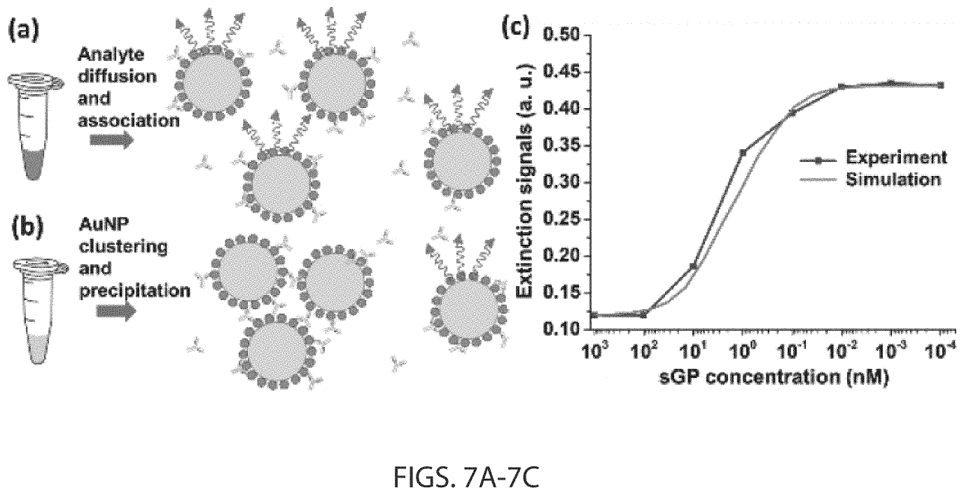

Embodiments of the present disclosure will also establish simple physical pictures and develop a mathematical model to understand the dynamic analyte-ligand-MNP binding and subsequent MNP precipitation process in antibody sensing. First, the characteristic time constants will be estimated to identify the reaction-determining steps during the sensing process, including the analyte diffusion, analyte-ligand binding, MNP diffusion, MNP clustering, and MNP precipitation (FIG. 7). The diffusivities of MNPs and analyte (proteins and antibodies) can be estimated from the Stokes-Einstein equation D=kT/(3πηd), where kT is the thermal energy, η is the solution viscosity (˜1.7×10⁻³ N·sec/m² for 20% glycerol in water), and d is the particle diameter. The diffusivity is estimated Dₐ˜6.7×10⁻¹¹ m²/s for a 5 nm protein and D_{NP}˜4.2×10⁻¹² m²/s for an 80 nm MNP. The diffusion length Lₐ for analyte to collide with MNPs can be further estimated, e.g., where Lₐ is ˜2 μm at low sGP concentration (<100 pM) but <100 nm at higher concentration (>1 μM). Therefore, the analyte diffusion time tₐ˜Lₐ²/Dₐ is found only 0.1 to 0.2 sec, much shorter than the experimentally observed assay time (˜3 hours, FIG. 6). On the other hand, given the high binding affinity of analyte-ligand complexes being detected, the association process of this complex is likely to be fast (e.g., G protein binds to GPCR receptors within ˜0.3 sec), thus also unlikely the limiting step.

In the meanwhile, the MNPs also diffuse, collide with and bind to each other, and accordingly form dimers and oligomers and even larger clusters. This dynamic process is rather complex, particularly considering the availability of multiple ligands on the MNP surface (estimated 120, 460, and 730 sites on 40 nm, 80 nm, and 100 nm diameter NPs). This multi-valent binding is crucial to the performance of this colorimetric, spectrometric, or electronic assay. First, it plays an important role in setting the dynamic range. The ultimate lower limit of detection (assuming long enough assay time and high enough binding affinity) could be perceived when each MNP is in average bound to a very small amount of analyte (e.g., smaller than one), and estimated ˜40 pM for the 80 nm MNPs, which is comparable to experimental analysis. The upper limit of detection could be estimated when the MNPs are completely saturated with the analyte (e.g., 460×0.036 nM or 16 nM for 80 nm MNPs), also comparable to but smaller than experimental values. This is reasonable considering a non-zero analyte concentration in solution when the dynamic association and dissociation processes reach balance. Second, a more accurate analysis must take into account the association/dissociation processes. Since the conjugation of ligands (detecting antibodies) on MNPs is through biotin-streptavidin binding that has a femto-molar dissociation constant far below the targeted detection range, the analyte-ligand binding should be the most critical. Their binding can be described as kₒₙ[MNP][A]=k_{off}[MNP·A] or [MNP·A]=k_{A}[MNP][A], where [A] and [MNP] are the free analyte and MNP concentrations, kₒₙ and k_{off} the association and dissociation constants, and k_{A}=kₒₙ/k_{off}=1/k_{D} the binding affinity. At equilibrium:

[MNP]ᵢₙᵢ=[MNP]+[MNP·A]+[MNP·2A]+[MNP·3A]+ . . . =[MNP]+k_{A}[MNP][A]+k_{A}²[MNP][A]²+k_{A}³[MNP][A]³+ . . . . (1)

[A]ᵢₙᵢ=[A]+[MNP·A]+2[MNP·2A]+3[MNP·3A]+ . . . =[A]+k_{A}[MNP][A]+2k_{A}²[MNP][A]²+3k_{A}³[MNP][A]³+ . . . . (2)

These equations allow the establishment of a mathematical model to predict the free MNP concentration and accordingly the extinction signals of the assay. Given that the higher-order clusters have much lower concentrations due to precipitation, [MNP] can be calculated and thus the extinction signals estimated from only the low-order oligomers. For an 80 nm MNP Ebola assay, this estimation was found in good agreement with experimental data (FIG. 7C). Third, the AuNP clustering would gradually cause sedimentation (or precipitation) due to gravitational force. The sedimentation time is estimated by t_{sed}=z/s·g, where z is the MNP precipitation distance (height of the solution in microcentrifuge tube), s is the sedimentation coefficient

$$s = \frac{{d}^{2} ( {ρ}_{M} - {ρ}_{w} )}{18 η}$$

and g is gravitational constant. Assuming a solution height of 5 mm, it was noticed that t_{sed} changes from 38 hours for a single 80 nm AuNP to 1.5 hours and 0.4 hour for a 400 nm and 800 nm AuNP clusters, respectively. This indicates that the NP aggregation and sedimentation can be the limiting factor of the assay speed. In addition, another time constant to consider is the ligand-analyte binding time τ=1/k_{off}. For Ebola sGP binding, a τ˜1.5 hour from k_{D}=4.63 nM and k_{off}=1.88×10⁻⁴ s⁻¹ was used. Interestingly, this is comparable to the theoretical sedimentation time, indicating that smaller AuNP aggregates may partly dissociate during sedimentation and return to the suspension, thus resulting in precipitation primarily in larger AuNP clusters as observed (FIG. 5B).

Embodiments of the present disclosure include sandwich and competitive assays for immunoglobulin and nAb detection. In accordance with these embodiments, the assays can include two or more sets of MNPs that each are conjugated with different ligands. By designing MNPs with different materials (e.g., gold and silver), sizes, and shapes, distinct color changes in multifunctional sensing can be used.

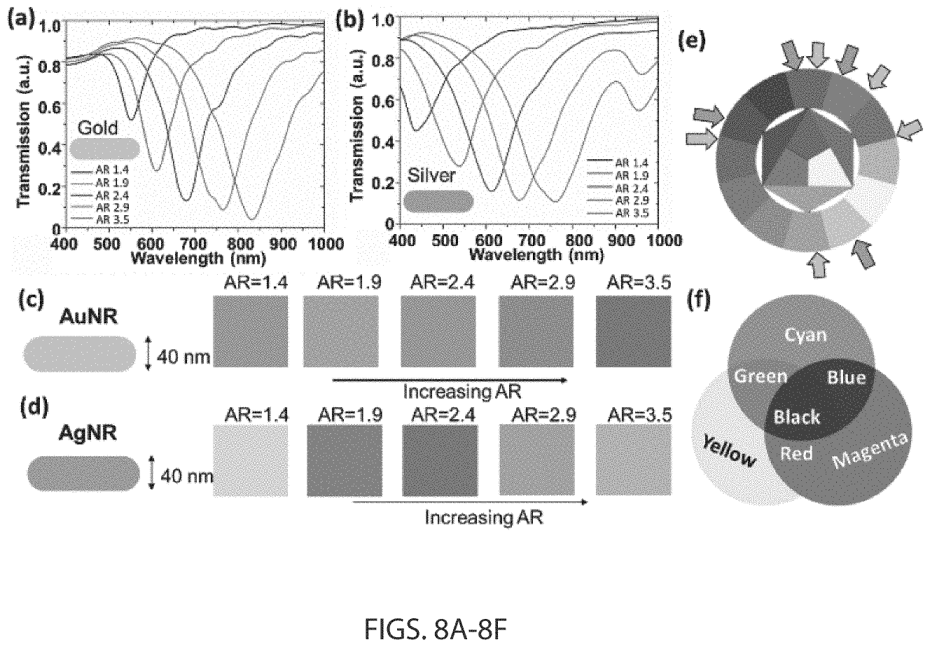

In some embodiments, the size of spherical MNPs modulates the suspension color (FIG. 6). However, such a modulation is rather small, making it less ideal for multi-analyte or multifunctional sensing by bare eyes. Thus, in some embodiments, gold nanorods (AuNRs) and silver nanorods (AgNRs) with different length-to-diameter aspect-ratios (ARs) can be used. The change in AR affects the dipole moment resulting from charge oscillation and accordingly the plasmonic resonance wavelengths, as shown from finite-difference time-domain (FDTD) simulation (FIGS. 8A-8B). Effectively, the suspension color can be tuned from blue to red and yellow (FIGS. 8E-8C (shown in greyscale)). Given the subtractive color-mixing scheme, AuNRs and AgNRs can be designed to have distinct color change upon mixing (FIGS. 8E-8F) for bare-eye readout of multiple analyte for each MNP reaction schemes.

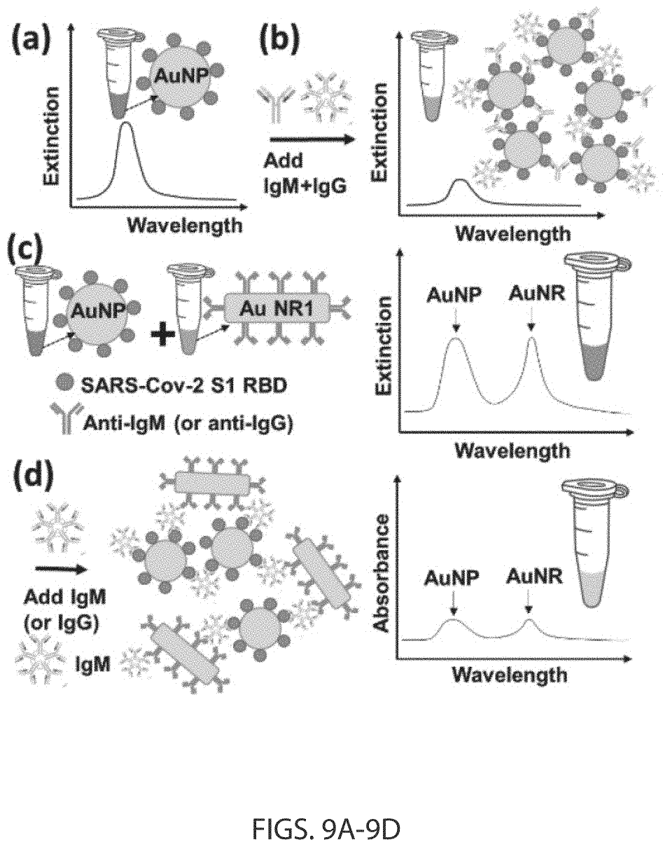

To establish a sandwich-type assay, two or more sets of MNPs will be surface-modified to conjugate immunoglobulin-specific anti-IgG (or anti-IgM and anti-IgA) antibodies and virus-specific RBD, for specific antibody detection (FIG. 9). The use of RBD-coated MNPs will enable binding to different types of immunoglobulins, which cause MNP aggregation and precipitation and accordingly drop in assay suspension color intensity (FIGS. 9A-9B To selectively detect IgG or IgM, a mixture of RBD-conjugated MNPs and anti-IgG (or anti-IgM) coated NRs (FIGS. 9C-9D) will be used. Upon mixing with IgG or IgM, clustering of NPs and NRs is expected to occur. This will reduce the extinction signals from both the NPs and NRs and thus decrease the assay suspension color intensity, which can be used for quantification.

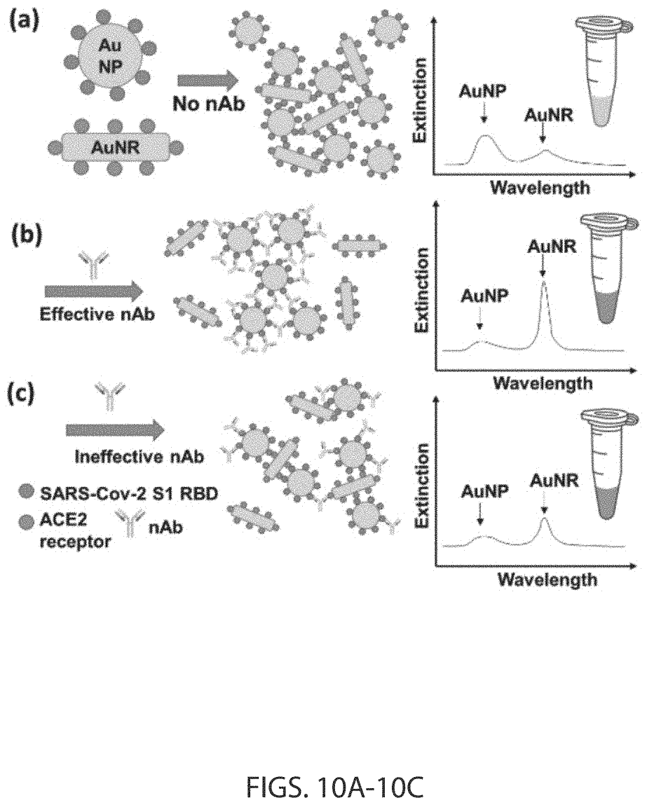

Additionally, a competitive assay will be used to evaluate the binding affinity and epitopes of nAbs. In some embodiments, two sets of heterogeneous MNPs that are surface-conjugated with RBD (or virus S1 protein) and human angiotensin-converting enzyme 2 (ACE2), respectively, will be used as the sensing assay. Mixing these two MNP suspensions without nAbs will lead to RBD-ACE2 binding and MNP clustering, resulting in a decreased suspension color intensity (saturation) and/or change in color (FIG. 10A), which is tunable by adjusting the initial RBD-ACE2 molar ratio and MNP geometries. Mixing nAbs together with the two suspensions will trigger competition between nAbs and ACE2-coated MNPs in binding of RBD (or S1 proteins), thus decreasing the amount of AuNP precipitation and subsequently modulating the suspension color change (FIGS. 10B-10C). The measurement of the optical extinction, therefore, can be used to quantify the antibody efficacy, which is dependent on the success of competition and affected by the binding affinity, epitopes, and concentrations of the nAbs to be tested.

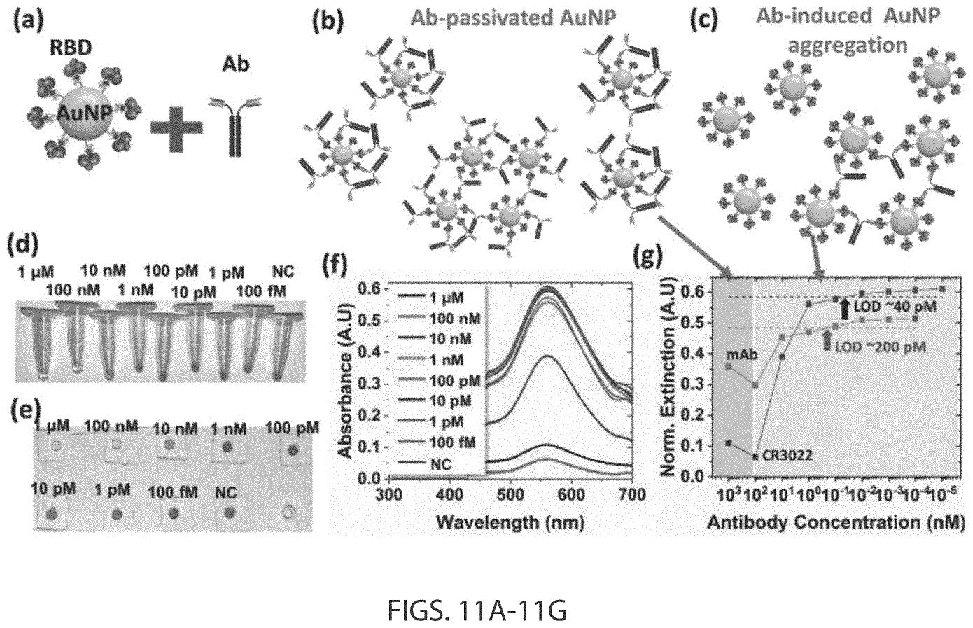

In addition, FIGS. 11A-11G shows exemplary results of RBD-binding antibody (Ab) testing. FIGS. 11A-11C show schematics of testing. Here RBD-functionalized 80 nm AuNPs were used to detect the two Abs by a centrifugation-accelerated method described herein. FIG. 11B shows a scheme of Ab in excess (Ab-passivated AuNP): some AuNPs fully covered with Abs are protected from aggregation. FIG. 11C shows a scheme of Ab not in excess (Ab-induced AuNP aggregation): Ab induces AuNP aggregation and extinction change. FIGS. 11D and 11E show optical images of RBD-AuNP and CR3022 (i.e., non-ACE2-competing antibody CR3022) mixtures in centrifuge tubes and PDMS plate. FIG. 11F shows measured optical extinction spectra of samples in FIG. 11E. FIG. 11G shows CR3022 and mAb (monoclonal antibody (mAb, Prosci Cat. No. 10-560)) sensing curve in PBS. The two terminal dots of each of the two curves (upper right on the graph) are negative controls (NC). The LOD of CR3022 (with a KD ˜6 nM in RBD binding) was found ˜40 pM, 5 times better than that of the mAb (LOD ˜200 pM, corresponding to KD ˜20 nM). Both curves display two concentration-dependent regimes: Ab-induced aggregation (green (shown in greyscale)) and Ab passivation (brown (shown in greyscale)). This confirms that Abs play two competing roles: they induce AuNP aggregation at low concentration but passivate the AuNPs and prevent aggregation when they are in great excess. The transition occurs around 100 nM to 1 uM.

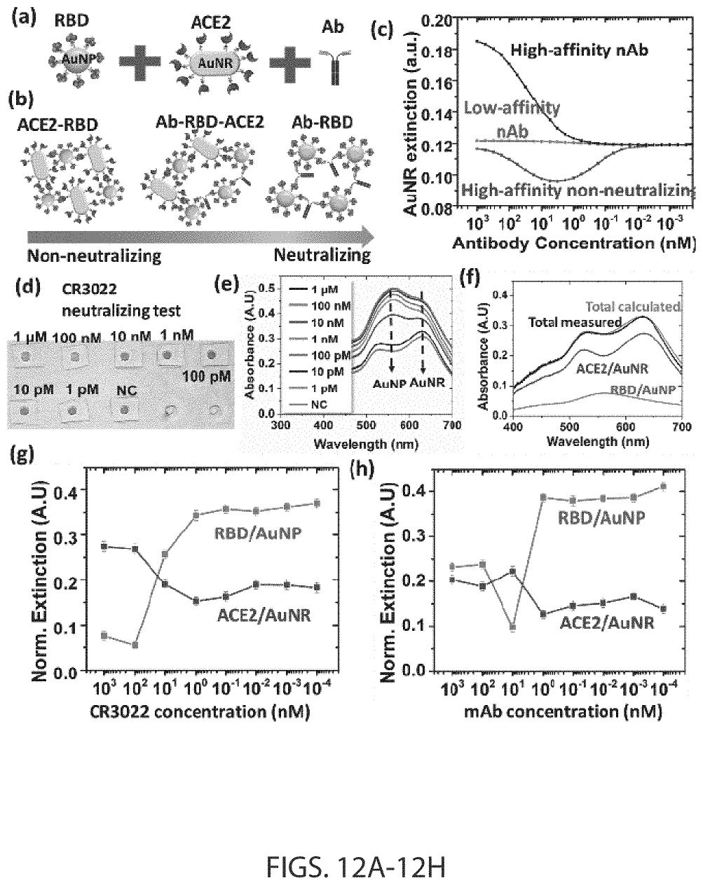

As a further illustration, FIGS. 12A-12H show exemplary data from neutralization testing. FIGS. 12A and 12B are schematics showing the binding of RBD/AuNP, ACE2/AuNR, and Ab. This could result in ACE2-RBD binding (non-neutralizing), ACE2-RBD-Ab binding (partial neutralizing), and Ab-RBD binding (effectively neutralizing). FIG. 12C shows a simulation, based on an established mathematical model combining three-body ligand binding kinetics and Smoluchowski coagulation equations, illustrating that the AuNR (ACE2) signals will deviate greatly for high-affinity nAb, low-affinity nAb, and high-affinity yet non-neutralizing Ab. Here the ACE2-RBD binding affinity K_{D}=15 nM is used as the threshold to differentiate high-affinity and low-affinity Abs. The amplitude is not calibrated to experimental conditions. FIGS. 11D and 11E show a PDMS well plate image and optical extinction in CR3022 neutralizing test, respectively. FIG. 12F shows the experimental optical extinction (black) in FIG. 12E at 1 μM Ab concentration can be fit as a sum of ACE/AuNR and RBD/AuNP signals, and is consistent with the calculated sum (grey). FIGS. 11G and 11H show ACE/AuNR and RBD/AuNP signals for CR3022 and mAb. The AuNR (ACE2) signals are found in both cases to increase at higher Ab concentration, indicating a level of protection of ACE2 proteins, i.e. neutralizing effect. Yet, in both cases the optical extinction did not reach original extinction without Ab (˜0.4), indicating only partial neutralization.

### 3. Detection Systems

Embodiments of the present disclosure also include sensing systems and corresponding kits incorporating the compositions and assays described herein. For example, in some embodiments, the systems and kits of the present disclosure include a device for measuring and/or detecting antibodies using the assays described herein. In some embodiments, systems will be capable of detecting and/or measuring SARS-CoV-2 antibodies with a time period of minutes.

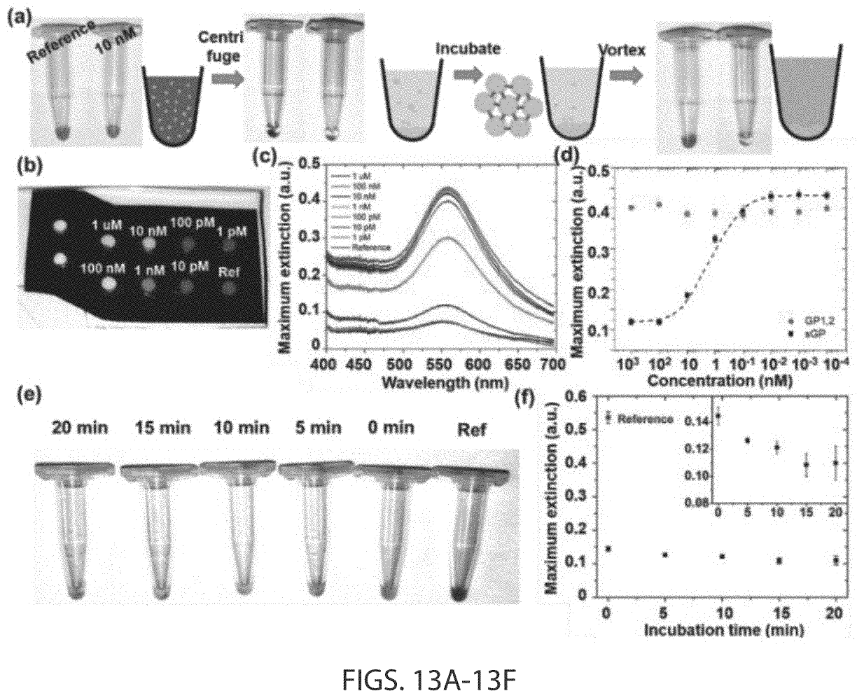

From theoretical and experimental analysis of the sensing mechanism described above, AuNP sedimentation time and the dissociation time are likely important factors affecting the assay time. From the estimation of the sedimentation time t_{sed}=z/s·g, the MNP precipitation distance z is an assay-relevant variable once the MNP size is defined. Therefore, one straightforward approach to improve the detection speed is to decrease z, which can be achieved by reducing the assay volume while keeping the concentration unchanged. However, there is a limit in volume reduction given presumably larger handling error at smaller volume. The use of centrifugation can be used to concentrate the AuNPs at the bottom of the container to minimize t_{sed} (FIG. 13).

In previous studies with Ebola sGP proteins, an extra brief centrifugation step was performed (3,500 rpm or 1,200×g, 1 min). The precipitation distance z was expected to accordingly drastically decrease from ˜4-5 mm to estimated 10-100 um (close packing of the AuNPs will result in <1 um height), e.g., ˜2 orders magnitude reduction. Then after a 20 minutes incubation, the assay colloid was thoroughly vortexed. This step was meant to resuspend the AuNP monomers that could have been physically adsorbed at the tube bottom without strong binding (FIG. 13A). From the PDMS well plate, the sGP could be identified by bare eyes down to ˜1 nM (FIG. 13B). From the extinction spectra of the supernatant (FIG. 13C), the maximum for each concentration was extracted and the standard curve was plotted (FIG. 13D). The extinction-concentration standard curve for the 20-min rapid detection was consistent with the measurement for 3-hour incubation (FIGS. 6E-6H), with comparable limit of detection (˜36 pM) and dynamic ranges (100 nM to 10 pM). In addition, tests for GP1,2 molecules specificity were performed, which is a homotrimer glycoprotein mainly found on virus membrane and transcribed from the same GP gene as sGP. Clearly, there was very little extinction signal change at different GP1,2 concentration, indicating a very good binding selectivity was maintained during this process.

Moreover, this rapid-detection method was used to detect 10 nM sGP at different incubation times, and it was found the color contrast was high enough that it can be resolved by naked eye right after resuspension by vortex-mixing without introducing additional incubation (FIG. 13E). This was consistent with the peak extinction signals normalized to incident light intensity (FIG. 13F), where the signal right after vortex-mixing (0.145) was completely distinguishable from the reference sample (0.536). The extinction signal was observed to gradually stabilize to 0.11 as incubation was extended to 20 min. Even including all the sample handling, such as pipetting, centrifugation, vortex, and readout (by naked eyes), the total detection time was reduced to within 5 minutes, or within 30 min if using a 20-min incubation. Such a rapid-detection method is particularly suitable for high-speed mass screening of large populations.

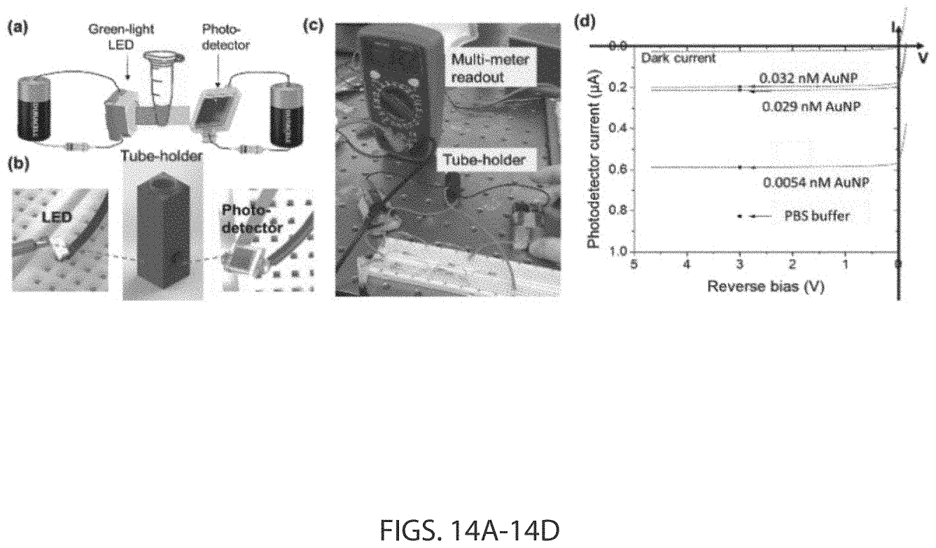

Embodiments of the present disclosure also include a portable and integratable electronic readout system. In some embodiments, a portable detector that quantifies MNP suspension color will be used to determine a precise readout. Here a pair of low-cost LEDs and photodetectors will be attached to a 3D-printed microcentrifuge tube holder for miniaturized system integration (FIG. 14A). The basic working principle is simple: a LED emits narrow-band light at the MNP extinction wavelength, which is strongly absorbed and scattered by MNPs in the centrifuge tube, and the transmitted light will be then collected by a photodetector and read out as either the photodetector current or voltage on a serial resistor. The technology has a few advantages compared to the spectroscopic readout. First, the LEDs, photodetectors, as well as other electronic components (such as batteries, resistors and ammeters, or voltmeters) are large-scale manufacturable and commercially available at very low cost. (For example, green LEDs cost $0.50 each and photodetectors cost $1.40 each.) This can significantly lower the cost of the sensing system and make it much more easily accessible. Second, these electronic components have very small foot-print (typically a few millimeters to one centimeter) and can be easily integrated into a portable and light-weight readout device. This will greatly facilitate its use in point-of-care applications. For example, the sizes of LEDs and photodetectors are comparable to the diameter of an electric wire welded to them (FIG. 14B). Further integration of multiple such LED/photodetector pairs is feasible onto printed circuit board for compact and multiplexed readout. Third, the electronic readout is much more accurate than bare-eye readout, and is accessible to anyone, including those who face challenges in color perception. Fourth, the electronic readout can be readily stored into computers or online database, saving time for data management and making the data available for long period of time.

In accordance with these embodiments, a black holder was 3D-printed that sung-fits microcentrifuge tubes on the top and has windows to mount the LED and photodetector to its sides (FIG. 14B). Two alkaline batteries were used to power the LED and bias the photodetector, and a multimeter was used to readout the voltage signal on a resistor in series with a photodiode that is reverse-biased (and thus producing a photocurrent insensitive to bias) (FIG. 14C). Tested with AuNPs at different molar concentration, it was found that such a simple system can readily distinguish the reference (PBS buffer without AuNPs, ˜0.83 μA), 0.0054 nM AuNPs (corresponding to sensing Ebola sGP proteins at 1 μM, ˜0.6 μA), 0.029 nM AuNPs (corresponding to sensing sGP proteins at 100 pM, ˜0.2 μA), and 0.032 nM AuNPs (corresponding to initial AuNP concentration without sGP proteins, ˜0.21 μA). These results clearly demonstrated that the portable electronic readout system can be adopted for SARS-CoV-2 sensing.

What is claimed is:

1. A composition comprising:
a first plurality of plasmonic metal nanoparticles (MNPs) having a SARS-CoV-2 antigen bound to its surface; and
a second plurality of MNPs having an anti-IgG and/or an anti-IgM binding moiety bound to its surface;
wherein the first and second pluralities of MNPs form a complex in the presence of a sample comprising a target antibody that recognizes the SARS-CoV-2 antigen.

2. The composition of claim 1, wherein the first and second pluralities of MNPs comprise a size and shape suitable for colorimetric, spectrometric, or electronic detection.

3. The composition of claim 1, wherein the SARS-CoV-2 antigen comprises an S1 subunit or the receptor binding domain (RBD) of the spike (S) protein, or a fragment thereof.

4. The composition of claim 1, wherein the composition further comprises the sample, and wherein the sample is obtained from a subject's bodily fluid.

5. A method of performing a colorimetric, spectrometric, or electronic assay using the composition of claim 1, the method comprising:
combining the first and second pluralities of MNPs with the sample from a subject; and
detecting an altered MNP extinction wavelength corresponding to the first and/or second pluralities of MNPs based on the presence or absence of the target antibody.

6. A system for performing the electronic assay of claim 5, the system comprising:
a receptacle for combining the first and second pluralities of MNPs with the sample from a subject;
a light source capable of emitting an MNP extinction wavelength corresponding to the first and/or second pluralities of MNPs; and
a photodetector capable of detecting transmitted light from the first and/or second pluralities of MNPs.

7. The system of claim 6, wherein the system further comprises a means for determining a voltage and/or current readout corresponding to the transmitted light detected by the photodetector.

8. A composition comprising:
a first plurality of plasmonic metal nanoparticles (MNPs) having a SARS-CoV-2 antigen bound to its surface; and
a second plurality of MNPs having a SARS-CoV-2 antigen binding moiety bound to its surface;
wherein the first and second pluralities of MNPs form a complex in the absence of a sample comprising a target antibody that recognizes the SARS-CoV-2 antigen.

9. The composition of claim 8, wherein the first and second pluralities of MNPs comprise a size and shape suitable for colorimetric, spectrometric, or electronic detection.

10. The composition of claim 8, wherein the SARS-CoV-2 antigen comprises an S1 subunit or receptor binding domain (RBD) of the spike (S) protein, or the S protein, or a fragment thereof.

11. The composition of claim 8, wherein the SARS-CoV-2 antigen binding moiety comprises the angiotensin-converting enzyme 2 (ACE2), or a fragment thereof.

12. The composition of claim 8, wherein the composition further comprises the sample, and wherein the sample is obtained from a subject's bodily fluid.

13. A method of performing a colorimetric, spectrometric, or electronic assay using the composition of claim 8, the method comprising:
combining the first and second pluralities of MNPs with the sample from a subject; and
detecting an altered MNP extinction wavelength corresponding to the first and/or second pluralities of MNPs based on the presence or absence of the target antibody.

14. A system for performing the electronic assay of claim 13, the system comprising:
a receptacle for combining the first and second pluralities of MNPs with the sample from a subject;
a light source capable of emitting an MNP extinction wavelength corresponding to the first and/or second pluralities of MNPs; and
a photodetector capable of detecting transmitted light from the first and/or second pluralities of MNPs.

15. A composition comprising a plurality of plasmonic metal nanoparticles (MNPs) having a SARS-CoV-2 antigen bound to its surface, wherein the plurality of MNPs form a complex in the presence of a sample comprising a target antibody that recognizes the SARS-CoV-2 antigen.

16. The composition of claim 15, wherein the plurality of MNPs comprise a size and shape suitable for colorimetric, spectrometric, or electronic detection.

17. The composition of claim 15, wherein the SARS-CoV-2 antigen comprises an S1 subunit or receptor binding domain (RBD) of the spike (S) protein, or a fragment thereof.

18. The composition of claim 15, wherein the composition further comprises the sample, and wherein the sample is obtained from a subject's bodily fluid.

19. A method of performing a colorimetric, spectrometric, or electronic assay using the composition of claim 15, the method comprising:
combining the plurality of MNPs with the sample from a subject; and
detecting an altered MNP extinction wavelength corresponding to the plurality of MNPs based on the presence or absence of the target antibody.

20. A system for performing the colorimetric, spectrometric, or electronic assay of claim 19, the system comprising:
a receptacle for combining the plurality of MNPs with the sample from a subject;
a light source capable of emitting an MNP extinction wavelength corresponding to the plurality of MNPs; and
a photodetector capable of detecting transmitted light from the plurality of MNPs.
